(19) United States
(12) Patent Application Publication
(10) Pub. No.: US 2023/0092872 A1
(43) Pub. Date: Mar. 23, 2023

(54) Method for promoting wound healing.

(71) Applicant: Phagelux Canada Inc., Montreal (CA)

(72) Inventor: Nancy Tawil, Cantley (CA)

(21) Appl. No.: 17/931,786

(22) Filed: Sep. 13, 2022

Related U.S. Application Data
(60) Provisional application No. 63/244,019, filed on Sep. 14, 2021.

(51) Int. Cl. A61L 26/00

(52) U.S. Cl. CPC A61L 26/0019; A61L 26/0066; A61L 2300/622; A61L 2300/624; A61L 2300/402; A61L 2300/41; A61L 2300/21; A61L 2300/204; A61L 2300/802

(57) ABSTRACT

A method of promoting wound healing in a patient, the method comprising applying on a wound a biodegradable amino-acid based polymer.

### FIELD OF THE INVENTION

The present invention relates to the general field of wound treatment and is more particularly concerned with a method for promoting wound healing.

### BACKGROUND

More than 40 million patients are afflicted with chronic wounds globally, with healthcare-associated costs exceeding $15 billions annually. Chronic wounds are characterized by damaged tissues with impaired healing processes within 4 weeks of standard of care treatment. The main factors responsible for impeded healing are age, impaired wound environment (i.e. increased production of metalloproteinases and proteases, impaired healing pathway activation, ischemia, and associated comorbidities such as immune deficiency, obesity, diabetes, and peripheral arterial disease. Moreover, bacterial infections complicate wound management by stimulating the production of proteolytic enzymes, altering the metabolic activity of cells, and limiting the diffusion of locally delivered drugs, thus impairing tissue regeneration.

Standard of care for chronic wound treatment consists of glycemic control, revascularization and optimization of the blood flow, removal of exudate, biofilm and necrotic tissue, and control over patients' co-morbidities. Despite this, chronic wounds remain a major impediment for the healthcare system with reduced effectiveness attributable to different factors such as unique healing process for each wound propelled by patient specific production of proteolytic enzymes making current all-purpose ideal dressings ineffective. Hence, much effort should be directed to the design of wound care products that account for the unique wound environment of each patient.

The primary goals of local wound management are the prevention of desiccation of viable tissue and the control of bacteria (ISBI Practice Guidelines Committee, 2016). Wound dressings are a central component of pressure injury care in order to keep the wound in a moist environment, thus promoting re-epithelialization and wound closure. Appropriate wound dressing should be based on goals and self-care abilities of the individual or caregiver and include considerations for diameter, shape and depth of the pressure injury; ability to keep the wound bed moist, nature and volume of wound exudate, condition of the tissue in the wound bed, condition of the peri-wound skin, and pain. Traditional wound dressings are comprised of gauzes, transparent films, foams, hydrogels, hydrocolloids, and hydroconductive dressings.

Gauzes are widely used and inexpensive but can re-injure the wound by causing trauma, mechanical debridement and pain when removed. Residues such as fibers and particles can remain in the wound causing activation of the immune system and granuloma formation. Moreover, wet to dry dressings can lead to vasoconstriction, hypoxia, patient discomfort, and bacterial contamination. Transparent films can simultaneously provide a moist wound environment, ensure gas exchange and prevent contamination from external bacteria without causing pain when removed. However, they are not recommended for highly exudative wounds. Foams are able to absorb large amounts of exudative wounds. Nonetheless, there is no evidence that foams alone can provide bacterial regression and improve healing rates in wounds.

Hydrogel-based wound dressings allow gas exchange, avoid patient's pain during their removal, enhance tissue granulation and are able to maintain a moist wound environment, which in turns promotes autolytic debridement. However, hydrogel dressings are less effective in facing bacteria contamination and are generally used in conjunction with anti-microbial agents and require frequent replacements.

Chitosan and alginate are naturally-derived and synthetic polymers and are used in hydrogels for wound healing applications. Nonetheless very few randomized clinical trials were able to demonstrate superiority of alginate compared to other commercial dressings. Chitosan is a polymer derived from chitin present in crustacean shell and suffer form high batch-to-batch variability affecting polymer average molecular weight and bioactive properties. Hydrogel dressings based on synthetic polymers show advantages over naturally derived polymers but have not been shown to actively participate to the healing process, therefore limiting their effectiveness as a stand-alone therapy.

Accordingly, there exists also a need for new methods to accelerate wound treatment. An object of the present invention is to provide such methods.

### SUMMARY OF THE INVENTION

The invention is concerned with compositions including amino-acid based polymers, for example in the form of microcapsules or nanocapsules, such as a Polyester Amide Urea (PEAU), a leucine-based poly ester amide polymer, or another amino acid based copolymer. Due to both groups, ester and amide, such polymers are biodegradable (ester group) and have good thermal stability and mechanical strength (amide group with strong intermolecular interactions). The incorporation of leucine, or other suitable amino acid, improves the biocompatibility of the polymer. The biodegradation rate of this polymer can easily be adjusted by changing its exact composition and molecular weight. When microcapsules are formed, the degradation rate of the microcapsules can be adjusted by controlling the size and thickness of the microcapsules.

Such a polymer is synthesized, in some embodiments, by interfacial polycondensation of the monomer L6, di-p-sulfonic acid salt of bis-(L-leucine)-1,6-hexylene diester with trisphogene/sebacoyl chloride with water/dichloromethane system. This method is fast, irreversible, involves two immiscible phases at room temperature and lead to high molecular weight polymer. Synthesis of the monomer L6 was executed in the presence of p-toluene sulfonic acid by condensation of L-leucine with 1,6-hexanediol in refluxed cyclohexane, because it is less toxic than solvents such as benzene. Purification includes recrystallization from water, filtration and drying under vacuum.

The formulations containing microcapsules are fabricated using a water-in-oil-in-water double emulsion-solvent, where the addition of the bacteriophages occurs in some embodiments in the secondary emulsion to minimize their exposure with the solvent dichloromethane (DCM). The DCM can also be replaced by an other suitable solvent, such as ethyl acetate, chloroform, or another organic solvent. It was found that, in some embodiments, there is no need to use a hardening tank during preparation of the microcapsules. Hardening tanks require dilution of the microcapsule preparation, for example by a factor of 5 or more. In addition to requiring additional processes to recover the microcapsules in the relatively large volume of liquid, use of hardening tank results in dilution of any component left in the aqueous phase in which the microcapsules are suspended.

Other polymers usable in the invention include:

A polymer selected from

(1) a poly (ester amide urea) wherein at least one diol, at least one diacid, and at least one amino acid are linked together through an ester bond, an amide bond, and a urea bond,

(2) a poly (ester urethane urea) wherein at least one diol and at least one amino acid are linked together through an ester bond, a urethane bond, and a urea bond,

(3) a poly (ester amide urethane urea) wherein at least one diol, at least one diacid, and at least one amino acid are linked together through an ester bond, an amide bond, a urethane bond, and a urea bond,

(4) a poly (ester amide urethane) wherein at least one diol, at least one diacid, and at least one amino acid are linked together through an ester bond, an amide bond, and a urethane bond,

(5) a poly (ester urea) wherein at least one diol and at least one amino acid are linked together through an ester bond and a urea bond, and

(6) a poly (ester urethane) wherein at least one diol and at least one amino acid are linked together through an ester bond and a urethane bond,

further wherein

the at least one diol is a compound of formula:

HO—R₁—OH, R₁ is chosen from C₂-C₁₂ alkylene optionally interrupted by at least one oxygen, C₃-C₈ cycloalkylene, C₃-C₁₀ cycloalkylalkylene,

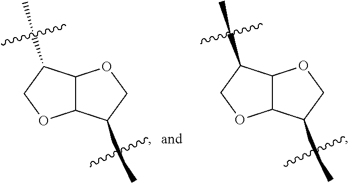 [SMILES: *[C@@H]1COC2C1OC[C@@H]2* ; *[C@@H]1COC2C1OC[C@H]2*]

the at least one diacid is a compound of formula:

HO—(CO)—R₃—(CO)—OH, R₃ is C₂-C₁₂ alkylene,
the at least one amino acid is chosen from naturally occurring amino acids and non-naturally occurring amino acid.

In some embodiments, the polymer is selected from

(1) a poly (ester amide urea) wherein at least one diol, at least one diacid, and at least one amino acid are linked together through an ester bond, an amide bond, and a urea bond,
(2) a poly (ester urethane urea) wherein at least one diol and at least one amino acid are linked together through an ester bond, a urethane bond, and a urea bond,
(3) a poly (ester amide urethane urea) wherein at least one diol, at least one diacid, and at least one amino acid are linked together through an ester bond, an amide bond, a urethane bond, and a urea bond, and
(4) a poly (ester amide urethane) wherein at least one diol, at least one diacid, and at least one

amino acid are linked together through an ester bond, an amide bond, and a urethane bond, wherein the at least one diol, at least one diacid, and at least one amino acid are as defined above.

In some more specific embodiments of the invention, the polymer is a poly (ester amide urea) comprising the following two blocks with random distribution thereof:

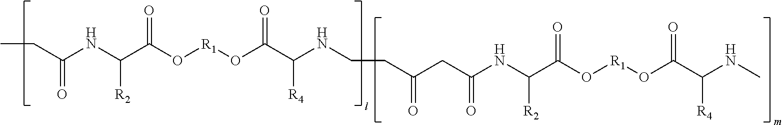

wherein

the ratio of l:m ranges from 0.05:0.95 to 0.95:0.05, l+m=1,

R₁ is chosen from C₂-C₁₂ alkylenes optionally interrupted by at least one oxygen, C₃-C₈ cycloalkylenes, C₃-C₁₀ cycloalkylalkylenes,

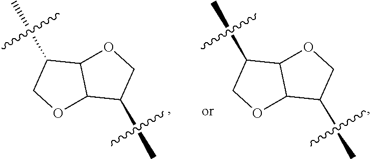

R₃ is C₂-C₁₂ alkylene,
R₂ and R₄ are independently chosen from the side chains of L- and D-amino acids so that the carbon to which R₂ or R₄ is attached has L or D chirality.

In some more specific embodiments of the invention, the polymer is poly (ester urethane urea) comprising the following two blocks with random distribution thereof:

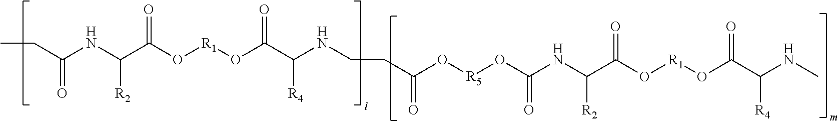

wherein
the ratio of l:m ranges from 0.05:0.95 to 0.95:0.05, l+m=1,
R₁ and R₅ are independently chosen from C₂-C₁₂ alkylenes optionally interrupted by at least one oxygen, C₃-C₈ cycloalkylenes, C₃-C₁₀ cycloalkylalkylenes,

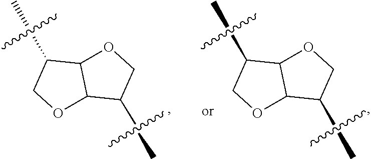

and
R₂ and R₄ are independently chosen from the side chains of L- and D-amino acids so that the carbon to which R₂ or R₄ is attached has L or D chirality.

In some more specific embodiments of the invention, the polymer is poly (ester amide urethane urea) comprising the following three blocks with random distribution thereof:

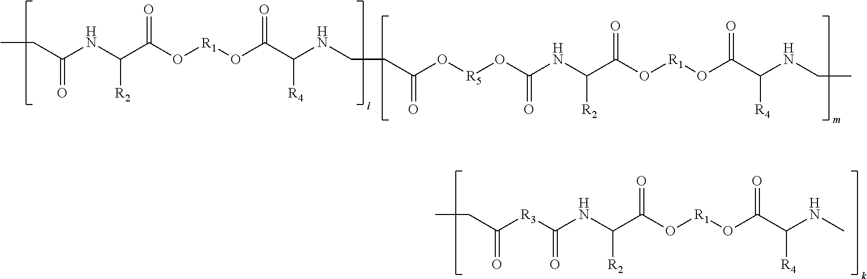

wherein
the ratio of l:m:k ranges from 0.05:0.05:0.90 to 0.90:0.05:0.05, l+m+k=1,
R₁ and R₅ are independently chosen from C₂-C₁₂ alkylenes optionally interrupted by at least one oxygen, C₃-C₈ cycloalkylenes, C₃-C₁₀ cycloalkylalkylenes,

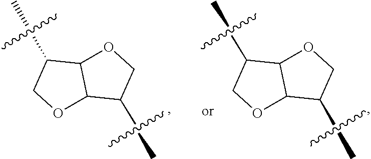

R₃ is C₂-C₁₂ alkylene, and
R₂ and R₄ are independently chosen from the side chains of L- and D-amino acids so that the carbon to which R₂ or R₄ is attached has L or D chirality.

In some more specific embodiments of the invention, the polymer is (ester amide urethane) comprising the following two blocks with random distribution thereof:

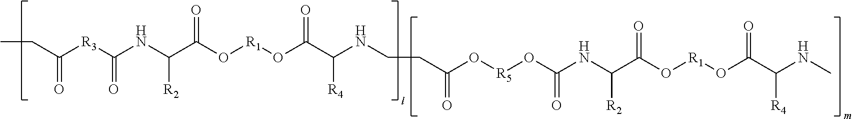

wherein

the ratio of l:m ranges from 0.05:0.95 to 0.95:0.05, l+m=1,

R₁ and R₅ are independently chosen from C₂-C₁₂ alkylenes optionally interrupted by at least one oxygen, C₃-C₈ cycloalkylene, C₃-C₁₀ cycloalkylalkylene,

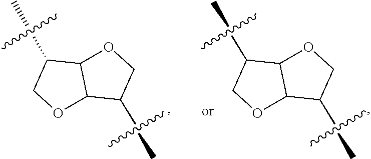

R₃ is C₂-C₁₂ alkylene, and

R₂ and R₄ are the same and selected from the side chains of L- and D-amino acids so that the carbon to which R₂ or R₄ is attached has L or D chirality.

In the above polymers, in some very specific embodiments of the invention, one or more of the following hold: R₁ is —(CH₂)₆—, R₃ is —(CH₂)₈—, or both R₂ and R₄ are the side chain of L-leucine.

Blends of the above-mentioned polymers are also usable in the preparation of the compositions of the present invention. More details regarding such polymers are provided in U.S. Pat. Nos. 10,772,964 and 10,849,944 issued respectively Sep. 15 and Dec. 1, 2020, the contents of which is hereby incorporated by reference in its entirety. The present application claims priority from U.S. provisional patent application 63/244,019 filed Sep. 14, 2021, the contents of which is hereby incorporated by reference in its entirety.

In some embodiments, the microcapsules are in suspension in a first aqueous suspension. In some embodiments, the microcapsules are full, that is devoid of a central cavity. In other embodiments, the microcapsules are hollow and encapsulate a second aqueous solution or suspension. Either or both of the first and second aqueous solutions/suspension may include polyvinyl alcohol, such as for example between 0.1% and 10% w/v of the polyvinyl alcohol. For example, and non-limitingly, the polyvinyl alcohol has a mean molecular weight of between 10 kDa and 400 kDa. In some embodiments, the polyvinyl alcohol is in a higher concentration in the second aqueous solution/suspension than in the first aqueous suspension. In some embodiments, the polyvinyl alcohol has a mean molecular weight of between 65 kDa and 90 kDa or between 10 kDa and 35 kDa.

In some embodiments, water-soluble salts are encapsulated in the polymer microcapsules, such as non-limitingly at least one salt selected from the group consisting of CaCO₃, Ca₃(PO₄)₂, MgCO₃, and Mg₃(PO₄)₂. The salt may be in the form of particles having a mean size between 2 μm and 15 μm, such as between 2 μm and 4 μm.

The polymer microcapsules have a mean size between 20 μm and 100 μm, for example between 20 μm and 50 μm.

The polymer is in some alternative embodiments in the form of nanocapsules, polymer sheets, or polymer powders, among others.

In some embodiments, the amino-acid based polymer has a molecular weight between 40 kDa and 105 kDa, for example between 40 kDa and 60 kDa.

In some embodiments, the composition also includes a poloxamer, such as non-limitingly poloxamer 407 in a concentration of between 10 and 30 percent. For example, the poloxamer has a mean molecular weight of between 9500 kDa and 15000 kDa.

In a broad aspect, there is provided a method of promoting wound healing in a patient, the method comprising: applying on the wound a composition including an amino-acid based polymer, wherein the amino-acid based polymer is selected from

(1) a poly (ester amide urea) wherein at least one diol, at least one diacid, and at least one amino acid are linked together through an ester bond, an amide bond, and a urea bond,
(2) a poly (ester urethane urea) wherein at least one diol and at least one amino acid are linked together through an ester bond, a urethane bond, and a urea bond,
(3) a poly (ester amide urethane urea) wherein at least one diol, at least one diacid, and at least one amino acid are linked together through an ester bond, an amide bond, a urethane bond, and a urea bond,
(4) a poly (ester amide urethane) wherein at least one diol, at least one diacid, and at least one amino acid are linked together through an ester bond, an amide bond, and a urethane bond,
(5) a poly (ester urea) wherein at least one diol and at least one amino acid are linked together through an ester bond and a urea bond, and
(6) a poly (ester urethane) wherein at least one diol and at least one amino acid are linked together through an ester bond and a urethane bond, further wherein

the at least one diol is a compound of formula:

HO—R₁—OH, R₁ is chosen from C₂-C₁₂ alkylene optionally interrupted by at least one oxygen, C₃-C₈ cycloalkylene, C₃-C₁₀ cycloalkylalkylene,

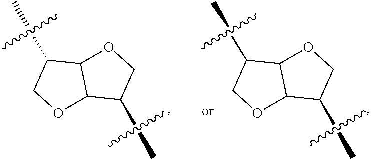

the at least one diacid is a compound of formula:

HO—(CO)—R₃—(CO)—OH, R₃ is C₂-C₁₂ alkylene,

the at least one amino acid is chosen from naturally occurring amino acids and non-naturally occurring amino acid.

There may also be provided a method wherein the amino-acid based polymer is a poly (ester amide urea) comprising the following two blocks with random distribution thereof:

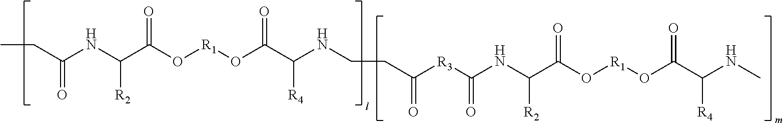

wherein
the ratio of l:m ranges from 0.05:0.95 to 0.95:0.05, l+m=1,
R₁ is chosen from C₂-C₁₂ alkylenes optionally interrupted by at least one oxygen, C₃-C₈ cycloalkylenes, C₃-C₁₀ cycloalkylalkylenes,

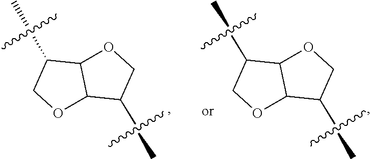

R₃ is C₂-C₁₂ alkylene,
R₂ and R₄ are independently chosen from the side chains of L- and D-amino acids so that the carbon to which R₂ or R₄ is attached has L or D chirality.

There may also be provided a method wherein the polymer is in the form of polymer microcapsules or nanocapsules.

There may also be provided a method wherein the microcapsules are suspended in a liquid, such as, non-limitingly, water or an aqueous solution.

There may also be provided a method wherein the composition is sprayed on the wound.

There may also be provided a method wherein the biodegradable amino-acid based polymer is devoid of anti-bacterial agents.

There may also be provided a method wherein the biodegradable amino-acid based polymer is devoid of bacteriophages or essentially devoid of bacteriophages. Compositions that are sequentially devoid of bacteriophages may include a very small quantity of bacteriophages, due for example to unwanted contamination, that would not be expected to provide any treatment benefit to the wound. For example, phage titers of less than 100 pFu/mL would be considered ineffective in wound treatment.

There may also be provided a method wherein the polymer is in the form of a film.

There may also be provided a method wherein the polymer is applied repeatedly on the wound.

There may also be provided a method wherein the polymer is applied daily on the wound.

There may also be provided a method the polymer is applied until the wound reaches a predetermined healing status. For example and non-limitingly, the predetermined healing status includes the formation of a scab or epithelial layer covering partially or entirely the wound or the formation of a dry wound that does not produce any exudate, among other possibilities.

There may also be provided a method wherein the wound is a chronic wound.

There may also be provided a method wherein the chronic wound is selected from the group consisting of diabetic foot ulcers, pressure ulcers, venous stasis ulcers, and ischemic ulcers.

There may also be provided a method wherein the composition further comprises at least one of an analgesic and an anti-inflammatory agent.

There may also be provided a method wherein the composition further comprises an at least one of ibuprofen, lidocaine, opioids, and cannabinoids.

There may also be provided a method wherein the microcapsules or nanocapsules consist essentially of the amino-acid based polymer.

There may also be provided a method wherein the microcapsules or nanocapsules consist of the amino-acid based polymer.

Other components may be added to the composition, either dissolved in the polymer or in suspension or solution in a fluid used to deliver the polymer. In some embodiments, metal ions, such as zinc or silver, are added to improve antibacterial activity. In other embodiments, one or more anti-microbial peptides (AMPs), such as non-limitingly Human Cathelicidin LL-37, Innate Defense Regulator 1018 peptide (IDR-1018), Human β-defensis (hBD-2 and hBD-3), Pexiganan, and Tiger17 peptide are added.

In some embodiments, Growth factors (Gfs), such as granulocyte-macrophage colony-stimulating factor (GM-CSF), basic fibroblast growth factor (bFGF), platelet derived growth factor (PDGF) and vascular endothelial growth factor (VEGF) are added.

In some embodiments, permeation enhancers, that facilitate permeation of the polymer in the tissues of the wound, such as polyethylene glycol (PEG), are added.

In some embodiments, stem cells, such as non-limitingly bone marrow-derived stem cells (BMSCs) and adipose-derived stem cells (ADSCs) are added.

PEAU and the other polymers described above are usable as ingredients in gauzes, transparent films, foams, hydrogels, hydrocolloids, and hydroconductive dressings that can be applied to the wound.

After application on the wound, the polymer (PEAU or other) degrades over time, releasing its component in the wound. If other components are included in the polymer, such as the other possible components that can be added to the formulation as described above, these components are also releases over time.

In other embodiments, the compositions described above are used to coat an implantable device that will be implanted in a patient. In yet other embodiments,

The composition can also be in the form of an implant usable in internal body sites and degrading gradually, for example over a period of 1 month to 1 year.

The compositions (polymer only or polymer with additional bioactive agents) may be dispersed or covalently bond in a patch, hydrogel, hydrocolloid or transparent film.

In some embodiments, the composition further includes cellular adhesion molecules (CAMs), antibiotics or antimicrobials and/or analgesics or anti-inflammatory agents.

Other objects, advantages and features of the present invention will become more apparent upon reading of the following non-restrictive description of preferred embodiments thereof, given by way of example only with reference to the accompanying drawings.

### BRIEF DESCRIPTION OF THE DRAWINGS

In the drawings:

FIG. 1, in scanning microscope micrographs, illustrates degradation of PEAU microcapsules by various agents;

FIG. 2, in photographs, illustrates typical result of treatment of non-infected wounds in placebo (top) and BACTELIDE treated (bottom) rats;

FIG. 3, in a histological H&E stain, illustrates a wound treated with BACTELIDE (magnification 20×);

FIG. 4 is a halftoned version of FIG. 3; and

FIG. 5 illustrates particle size distribution in a microcapsule formulation usable to perform the proposed method.

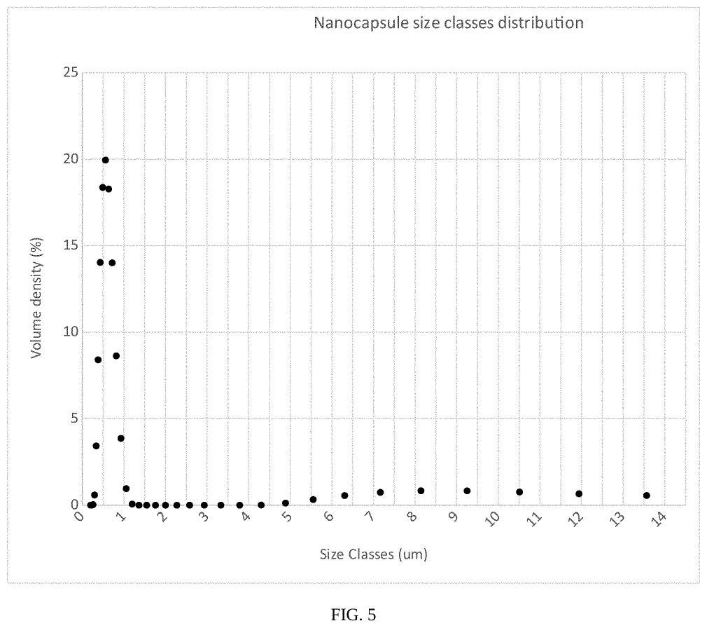

### DETAILED DESCRIPTION

The examples below use a polymer referred to as PEAU. This polymer is a poly (ester amide urea) comprising the following two blocks with random distribution thereof:

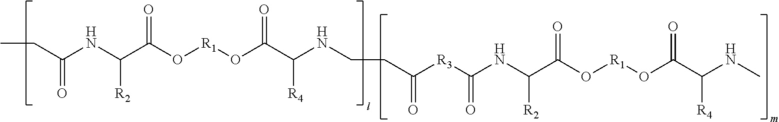

wherein
the ratio of l:m ranges from 0.05:0.95 to 0.95:0.05, l+m=1,
R₁ is chosen from C₂-C₁₂ alkylenes optionally interrupted by at least one oxygen, C₃-C₈ cycloalkylenes, C₃-C₁₀ cycloalkylalkylenes,

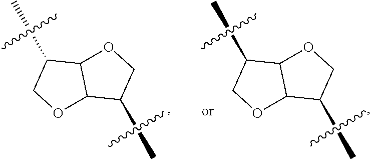

R₃ is C₂-C₁₂ alkylene,
R₂ and R₄ are independently chosen from the side chains of L- and D-amino acids so that the carbon to which R₂ or R₄ is attached has L or D chirality.

The more specific polymer referred to in the examples is the polymer wherein R₁ is —(CH₂)₆—, R₃ is —(CH₂)₈— and both R₂ and R₄ are the side chain of L-leucine. These polymers are referred to hereinbelow in abbreviated form as (8L6)ₗ-(1L6)ₘ.

The amino-acid based polymers and microcapsules may be prepared as follows, although other preparation methods are within the scope of the invention. PEAU is synthesized, in some embodiments, by interfacial polycondensation of the monomer L6, di-p-sulfonic acid salt of bis-(L-leucine)-1,6-hexylene diester with trisphogene/sebacoyl chloride with water/dichloromethane system. This method is fast, irreversible, involves two immiscible phases at room temperature and lead to high molecular weight polymer. Synthesis of the monomer L6 can be executed in the presence of p-toluene sulfonic acid by condensation of L-leucine with 1,6-hexanediol in refluxed cyclohexane. Purification includes recrystallization from water, filtration and drying under vacuum. The compositions containing microcapsules are fabricated using a water-in-oil-in-water double emulsion-solvent, where the addition of the bacteriophages occurs in some embodiments in the secondary emulsion to minimize their exposure with the solvent dichloromethane (DCM). The DCM can also be replaced by an other suitable solvent, such as ethyl acetate, chloroform, or another organic solvent. Polymer films may be manufacturing by spraying the polymer solution and drying. Powders may be obtained by grinding films or microcapsules, among other possibilities.

While evaluating in-vivo toxicity of the PEAU polymer, it was discovered that, unexpectedly, the PEAU polymer promoted wound healing. Indeed, application of a PEAU polymer formulation including bacteriophages led to wounds that healed faster than a control, even in sterile conditions in which the bacteriophages are expected to have no influence. It is also expected that the other amino-acid based polymers described in the present document would have similar effects based on their similar chemistry. Accordingly, using these polymers for wound treatment, even in the absence of bacteriophages would promote faster healing compared to the current standard of care.

More specifically, a study included a total of 44 rats, 40 of which were selected for test or control article treatment and randomized into groups based on weight and sex. Animals were microchipped for body temperature monitoring. One uniform size full-thickness excisional wound was created using a sterile template (16 mm diameter) and a surgical blade at the lateral/dorsal region. Baseline wound measurements and temperatures were taken. A predetermined volume of test (referred to as BACTELIDE herein, the composition of which is detailed below) or control (physiological buffer) (230-250 mL) was applied to the wound site by one actuation of a spray bottle pump. The spray was allowed to sit undisturbed for approximately five minutes. The site was covered with Mepilex® Lite foam dressing, and a secondary non-occlusive dressing. After a 24±2 hours exposure, the wound was measured and the treatment was reapplied. The application or re-application and wound measurement were performed for 28 consecutive days beginning on Day 1. At the Day 29 timepoint 20 animals had blood collected and then were euthanized. Tissues were collected, with a select list processed for histopathology evaluation. After a two-week recovery phase, blood was collected and animals were euthanized. Tissues were collected, with a select list processed for histopathology evaluation.

The BACTELIDE composition included a mixture of 14 lytic bacteriophages. These phages were encapsulated into a biodegradable polyester amide) urea (PEAU) co-polymer in the form of microcapsules, and delivered by a dosage-metered spray bottle and pump. Bactelide includes the microcapsules in suspension in a mixture including 17.33 mg/mL PEAU, 10 mg/mL PVA, 0.5844 mg/m: NaCl: 12037 MgSO₄, and 0.6057 mg/mL TRIS HCl.

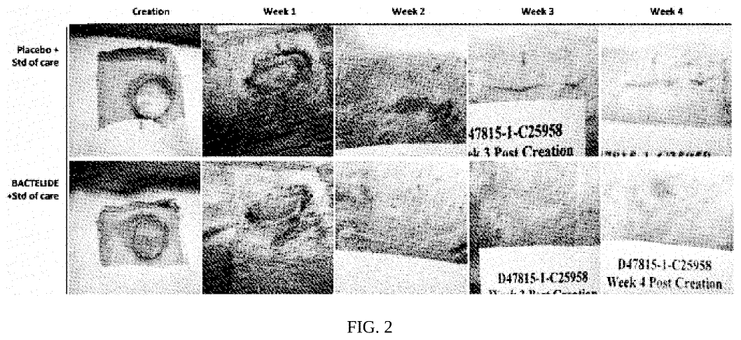

There were no adverse findings in the histopathology data of the skin or axillary lymph nodes attributed to BACTELIDE. The findings present were as expected following surgical wound creation and wound treatment. Under the conditions of this full-thickness wound-healing study and based on the Irritant Rank Score, BACTELIDE was considered a non-irritant when compared with the control (PBS only) at day 29 and day 43±1, and a moderate irritant compared to untreated skin at day 29 and day 43±1. The moderate irritant status compared to untreated skin was as expected, because the untreated skin contained no foreign material and had not received a surgical wound, and so only exhibited background findings. Wounds of male and female animals enrolled in BACTELIDE groups were fully healed prior to the wounds of male and female animals enrolled in control groups. All control male animals were fully healed by day 23 as compared to 18 days in the males treated with BACTELIDE; all female animals in the control group were fully healed by day 20 as compared to day 15 in the BACTELIDE. Rate of wound healing in the BACTELIDE test group was significantly higher 16.88% faster healing on day 7 and reaching 19.18% faster healing on day 10 for all groups combined. FIG. 2 illustrates typical result of treatment of non-infected wounds in placebo (top) and BACTELIDE treated (bottom) rats. Faster healing is clearly shown (middle column), as well as reduction in scar visibility (last column).

It is hypothesized that PEAU contributes to wound healing by utilizing proteases in the wound environment that are implicated in chronic wound pathophysiology. For example, chronic wounds (diabetic foot ulcers, pressure ulcers, venous stasis ulcers, and ischemic ulcers) are characterized by persistent inflammatory stimuli such as repeated trauma, ischemia, or low-grade bacterial contamination. In all these wounds, including pressure ulcers, the skin barrier is broken and bacterial colonization occurs and stimulates inflammatory cells such as neutrophils and macrophages to enter the wound. These activated inflammatory cells then secrete inflammatory cytokines such as TNF-α and IL-1 (which synergistically increase production of MMPs while reducing synthesis of TIMPs). The elevated protease secretion (MMPs, elastase, plasmin) degrades the ECM which interferes with cell migration and connective tissue deposition. Proteases also degrade growth factors and their target cell receptors which further limits the progression of the wound healing by eliminating the mediators of the cascade. Entry into the repair phase is thereby impaired, and the wound fails to heal. This differs from acute wounds in that there is a limited pro-inflammatory stimulus rather than ongoing stimulation as proposed in chronic wounds.

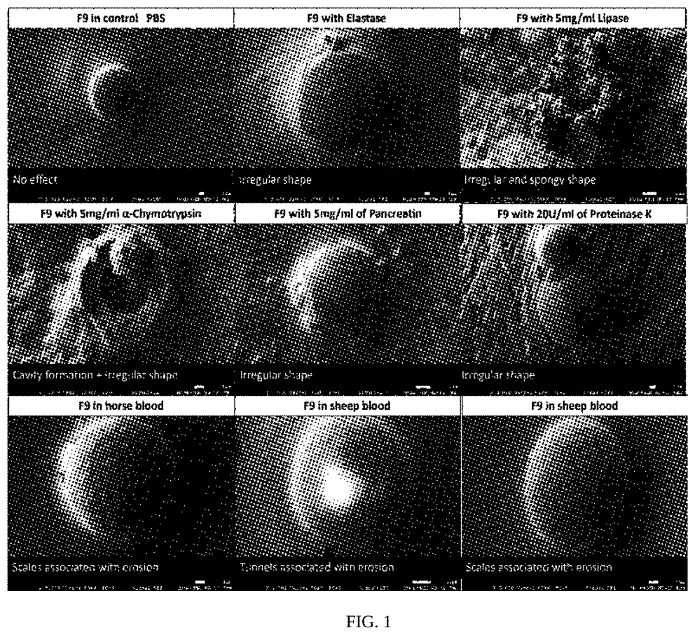

PEAU utilizes proteases in the wound environment to enzymatically degrade its constituent chains. PEAU has been shown to degrade following the action of proteases such as MMPs and elastases. Decreasing the amount of proteases in the environment is known to promote wound healing. FIG. 1 illustrates the enzymatic biodegradation of PEAU microcapsules through scanning electron micrographs of PEAU microcapsules in the presence of PBS, elastase, lipase, α-chymotrypsine, pancreatin, proteinase K, horse blood, and sheep blood.

In addition, the electrical properties of the side chain residues of PEAU positively impact the interaction of phages and the supported cells and promote wound healing. Positively charged PEAs, especially cationic 1-Lys-derived PEAs, provide a basis for materials to obtain relatively better biological properties, by interacting electrostatically with negatively charged cell membranes, thus promoting cellular internalization, cell adhesion, survival, growth, proliferation, differentiation, and migration for trauma repair and function reconstruction of tissues/organs. An additional secondary benefit of using polyester amides is that the degradation products (zwitterionic amino acids and diols, and relatively weak fatty di-acids) activate production of host growth factors and thus accelerate wound healing.

An in vivo full thickness study was performed on rats, which showed more efficacious tissue reactions for BACTELIDE than control, mediated in part by increased macrophage activation and stimulation of endothelial cell and smooth muscle cell proliferation. This is followed by an increase in myofibroblast population and neovascularization. The tissue deposition results in a collagen formation with fibrous band filling the surgically created wound in the dermis, subcutis, and panniculus muscle, covered with intact epidermis and merging with the pre-existing dermal collagen to either side. Stimulation of the tissue response was visualized by microcapsules embedded in the deep extent of the scar tissue, parallel to the skin surface surrounded by multifocal to coalescing small dense aggregates of macrophages (mild) and multinucleated giant cells (mild to moderate). Of interest is the presence of hypertrophic epidermidis in the control groups and its absence from the BACTELIDE treated group. Moreover, the Average Tissue Response Score (ATRS) was notable different between test article (males: 3.33; females: 4.66) and control (males: 5.66; females: 4.66) for the Day 43 groups, observing a lower score for specimen subjected to test article. These observations are likely to be an indication of the role of PEAU in immune stimulation and wound healing.

It is hypothesized that a beneficial effect of polyester amides is that the degradation products (zwitterionic amino adds and diols, and relatively weak fatty diacids) activate production of host growth factors and thus accelerate wound healing. An in vivo full thickness study showed a more efficacious tissue reactions than control mediated in part by increased macrophage activation and stimulation of endothelial cell and smooth muscle cell proliferation. This is followed increase in myofibroblast population and neovascularization. The tissue deposition results in a collagen formation with fibrous band filling the surgically-created wound in the dermis, subcutis, and panniculus muscle, covered with intact epidermis and merging with the pre-existing dermal collagen to either side. Stimulation of the tissue response was visualized by microcapsules embedded in the deep extent of the scar tissue, parallel to the skin surface surrounded by multifocal to coalescing small dense aggregates of macrophages (mild) and multinucleated giant cells (mild to moderate).

Amino acid-based biodegradable polymer encapsulation of active ingredients provides site of action delivery for increased bioavailability, prolonged release, and better compliance. BACTELIDE is non-toxic, non-irritants, biocompatible, hemocompatible, non-genotoxic, and does not exhibit in vivo local or systemic toxicity. BACTELIDE has been shown to promote tissue response, increase healing, and penetrate in deeper layers of the dermis. BACTELIDE allows for both superficial epidermis prolonged release and penetration promotion in the dermis PEAU biodegrades evenly and is stable at room temperature. PEAU and the other polymers described herein can be formulated into sprays, patches, thin films, powders, and incorporated into hydrogels, creams or suspensions, which are believed to promote also wound healing as described in the present example.

PEAU has several advantages over non-degradable polymers as well as degradable polymers such as PLLA and PLGA. Amino acid-based biodegradable polymers (AABBPs) are entirely composed of non-toxic building blocks such as naturally occurring amino acids and fatty diols and dicarboxylic, acids. These compounds contain hydrolysable ester bonds at a monomer stage, which when incorporated into the polymeric backbones are responsible for the biodegradation of the polymers.

AABBPs have many advantages over biodegradable polyesters (PEs) (polyglycolic and polylactic acids)) and their copolymers, including:

Polycondensation synthesis without using any toxic catalyst;
Higher hydrophilicity and, hence, better compatibility with tissues;
Longer shelf-life;
A wide range of desirable mechanical properties—from viscose-flow to strong materials with modulus of elasticity up to 6.0 gPa;
A variable hydrophobicity/hydrophilicity balance suitable for constructing devices suitable for sustained/controlled drug release;
An erosive mechanism and in vitro biodegradation rates ranging from 10-3 to 10-1 mg/(cm2·h) that can be regulated by the addition of enzymes;
The vast majority of AABBPS are amorphous and biodegrade completely and evenly;
Excellent adhesion to plastic, metal, and glass surfaces.

In addition, it has been shown that polyester degradation by-products are acidic resulting in undesirable side-effects at a cellular level, which limits their use as functional tissue engineering scaffolds. In contrast to polyesters, AABBPs degradation bi-products are less acidic and more biocompatible. For example, hydrolysis products of polyester amide)s are neutral (zwitterionic) amino acids, readily metabolizable diols, and weak fatty acids. Another example are the biodegradation products of poly(ester urethane)s and poly(ester urea)s which are naturally, occurring physiological compounds such as CO2, hydrophobic amino acids, and diols. Polyester amides are known to be biocompatible, hemocompatible, and are used in stent-based local drug delivery.

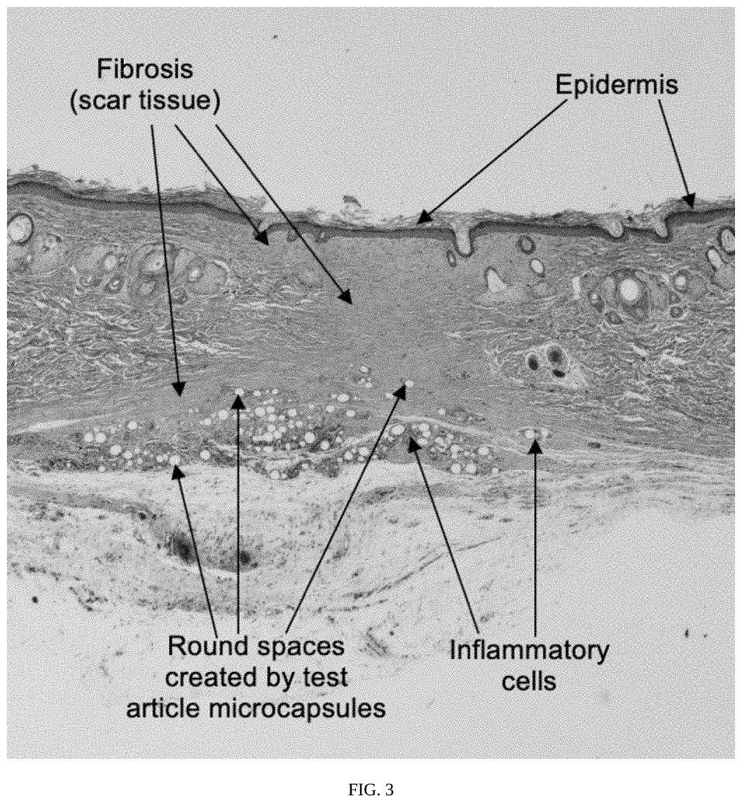

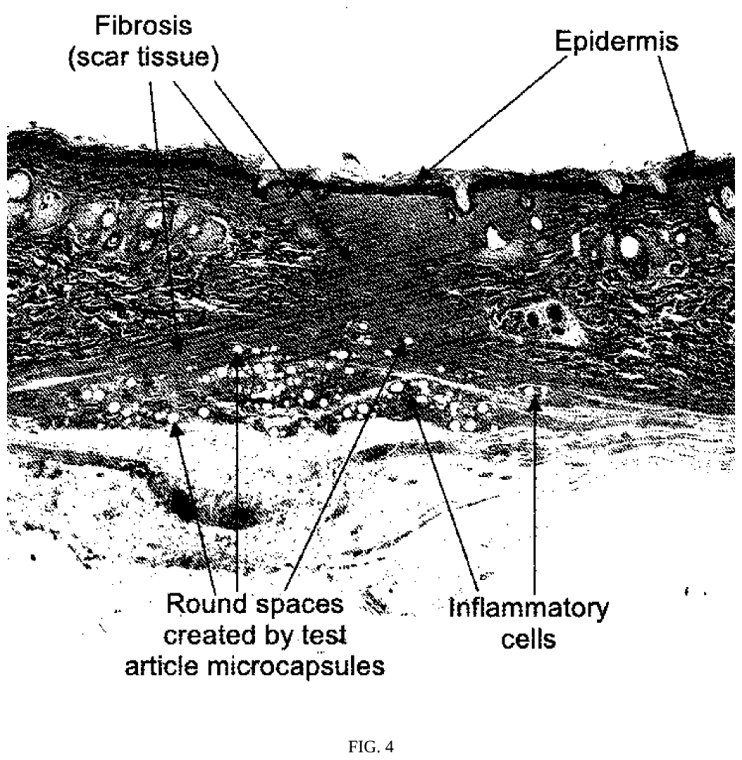

When used, microcapsules are particularly advantageous for treatment as they penetrate deeply in the dermis, enhancing the above-describe properties in the tissue, and are not present only at its surface. FIG. 3 clearly shows the round empty spaces left deep in the tissue by microcapsules in a histological H&E stain of a wound treated with BACTELIDE, which illustrates deep penetration. Such empty spaces are absent from controls. FIG. 4 is the same image processed by halftoning for better reproducibility in the issued patent.

### Toxicity Studies

Genotoxicity and mutagenicity assays according to the ISO 10993-3:2014 standard were performed to evaluate the mutagenic and carcinogenic potential of the extraction product of the PEAU polymer and the extraction product of the microcapsules (to assess the mutagenic potential of leachable substances or residues). Appropriate sample preparation procedures were done according to ISO 10993-3:2014(E) decision tree. The mutagenicity potential of the different samples was evaluated using the Ames modified ISO assay and the Pour plate Ames assay (with and without metabolic activation).

The following table shows that polymer extracts, and microcapsules extracts has no mutagenic potential (when we choose a significance level of 0.01).

TABLE 1
Results of the Ames test of undiluted (CC) «free
PL-03-BM», polymer extracts, and microcapsules
extracts expressed as Negative (N) or Positive (P)
mutagenic potential on the full battery of bacterial strains.

| | Polymer | | | | Microcapsules | | | |
|---|---|---|---|---|---|---|---|---|
| | extracted in | | Polymer | | extracted in | | Microcapsules | |
| Test | saline | | extracted in | | saline | | extracted in | |
| condi- | solution | | PEG400 | | solution | | PEG400 | |
| tion a | −S9 b | +S9 c | −S9 b | +S9 c | −S9 b | +S9 c | −S9 b | +S9 c |
| TA97a | N | N | N | N | N | N | N | N |
| TA98 | N | N | N | N | N | N | N | N |
| TA100 | N | N | N | N | N | N | N | N |
| TA1535 | N | N | N | N | N | N | N | N |
| WP2 | N | N | N | N | N | N | N | N |

a the results for undiluted (CC) samples were the same as the diluted ones (D1 → D4)
b without metabolic activation
c with metabolic activation

ISO 10993-18:2020 and ISO/TS 21726:2019 leachable studies were performed for BACTELIDE. Solutions of BACTELIDE were collected for analysis via the spraying. The extracts were analyzed by gas chromatography-mass spectrometry (GC-MS) for volatile to semi-volatile compounds, by liquid chromatography-mass spectrometry (LC-MS) for semi-volatile to nonvolatile compounds, and by inductively coupled plasma mass spectrometry (ICP-MS) for elemental (metal and other) components. After analysis, results were reported based on the evaluation criteria listed in the results section below. GC-MS based on the drug product extraction chromatographic data showed that five reportable compounds were identified in the test article extracts. 2-(N,N-Diethylamino)ethyldecyl maleate (CAS #184874-09-7) was the most abundant. Regarding LC-MS results, thirty-nine reportable compounds were identified in the test article extracts in positive ion mode. Glycerol dipentadecanoate (CAS #121957-69-5) was the most abundant. Thirty-six reportable compounds were identified in the test article extracts in negative ion mode. 3-[4-[[4-(2-Oxiranylmethoxy)phenyl]methyl]phenoxy]-1,2-propanediol didodecanoate (CAS #not given) was the most abundant. ICP-MS based on the drug product extraction elemental qualitative scan led to the identification of fifteen elements in the test article extracts. The most abundant element was magnesium.

A toxicological evaluation to assess the safety of the results reported, as they relate to the duration of exposure and nature of contact of the device, was performed to complete analysis of risk. A toxicological evaluation of leachable chemicals was performed to support safety and biocompatibility of the BACTELIDE formulation during its intended use in accordance with ISO 10993-1:2018, Biological Evaluation of Medical Devices, Part I: Evaluation and Testing Within a Risk Management Process (15010993-1, 2018); and FDA Guidance, Use of International Standard ISO 10993-1, “Biological evaluation of medical devices—Part 1: Evaluation and testing within a risk management process” (FDA, 2020). The safety question addressed in this risk assessment is whether intermittent, long term patient exposure to the microcapsule spray and levels of leachables from the test article could produce unacceptable human health risks, including carcinogenic and systemic non-carcinogenic risks.

In order to assess potential health risks posed by the test article, worst-case exposure estimations, including daily exposure and 100% bioavailability, were used to assess potential health risk. Solutions of drug product as contained in the submitted vials were collected for analysis via the spray actuation of the container and analyzed by GC-MS for volatile to semi-volatile compounds, by LC-MS for semi-volatile to nonvolatile compounds, and by ICP-MS for elemental (metal and other) components. All identified chemicals were assessed for risk.

The MOS values for all chemicals (or groups of similar chemicals) and elements identified across the analytical methods ranged from 3 to 1340000. A few chemicals had calculated “near-1” (i.e. ≥1 but <10) MOS values. As a group, the 3-[4-[[4-(2-Oxiranylmethoxy)phenyl]methyl]phenoxy]-1,2-propanediols had the lowest MOS value of three (3). However, the POD was based on body weight loss and elevated serum cholesterol. The gross findings and serum chemistry were not associated with adverse histopathology. Coupled with the IARC class 3 designation, the severity is interpreted as low and an acceptable exposure risk. Two chemicals (1,5-Dihydro-4-hydroxy-5-(phenylmethylene)-2H-pyrrol-2-one related compound and Dihydroxyphenyl trihydroxy-mono-carboxyacetylhexoside benzopyran-4-one) had MOS values of 5. Their margins of safety were calculated using the ICH M7 less than 1 year TTC of 0.33. Since exposure to product is intermittent through 56 days and because derivation of TTC levels is based on extrapolation from known compounds, the resulting MOS values are inherently conservative, which potentially overestimates the level of risk identified from the MOS calculations. Overall, there were no chemicals identified by GC-MS, LC-MS, or ICP-MS at levels of toxicological concern.

The risk of adverse health effects resulting from exposure to a material/product is dependent, among other factors, on the toxicological profiles of the chemical constituents and the nature and duration of contact. This study demonstrates acceptable worst-case margins of safety in the adult patient population for all identified chemicals, chemical groups, and elements from the PEAU microcapsule spray.

### Nanocapsules

PEAU and the other polymers described hereinabove can be provided in the form of nanocapsules to treat the wounds, instead of the microcapsules. Nanocapsules have be prepared as follows. 30 mL of PVA 10% was poured in a 100 mL beaker. The first emulsion was prepared by adding 1.2 mL of Kolliphor 1188 1% in 12 mL PEAU in DCM (12.5% (w/v)) and setting the homogenizer at 35,000 rpm for 15 min on ice. The second emulsion was prepared by adding the first emulsion in the beaker containing the PVA and homogenizing as described above. Finally, DCM was evaporated through overnight magnetic bar stirring. The particle size distribution is illustrated in 5. D10, D50 and D90 particle size distributions were respectively 0.4053, 0.5759 and 0.9283 nm. Therefore, a particle size distribution with a D90 of less than 1 nm was obtained. Nanoparticles are smaller and therefore have more permeation in the wound tissues than microparticles.

Although the present invention has been described hereinabove by way of exemplary embodiments thereof, it will be readily appreciated that many modifications are possible in the exemplary embodiments without materially departing from the novel teachings and advantages of this invention. Accordingly, the scope of the claims should not be limited by the exemplary embodiments, but should be given the broadest interpretation consistent with the description as a whole. The present invention can also be modified, without departing from the spirit and nature of the subject invention as defined in the appended claims.

What is claimed is:

1. A method of promoting wound healing in a patient, the method comprising: applying on the wound a composition including an amino-acid based polymer, wherein the amino-acid based polymer is selected from
(1) a poly (ester amide urea) wherein at least one diol, at least one diacid, and at least one amino acid are linked together through an ester bond, an amide bond, and a urea bond,
(2) a poly (ester urethane urea) wherein at least one diol and at least one amino acid are linked together through an ester bond, a urethane bond, and a urea bond,
(3) a poly (ester amide urethane urea) wherein at least one diol, at least one diacid, and at least one amino acid are linked together through an ester bond, an amide bond, a urethane bond, and a urea bond,
(4) a poly (ester amide urethane) wherein at least one diol, at least one diacid, and at least one amino acid are linked together through an ester bond, an amide bond, and a urethane bond,
(5) a poly (ester urea) wherein at least one diol and at least one amino acid are linked together through an ester bond and a urea bond, and
(6) a poly (ester urethane) wherein at least one diol and at least one amino acid are linked together through an ester bond and a urethane bond,
further wherein
the at least one diol is a compound of formula:
HO—R₁—OH, R₁ is chosen from C₂-C₁₂ alkylene optionally interrupted by at least one oxygen, C₃-C₈ cycloalkylene, C₃-C₁₀ cycloalkylalkylene, 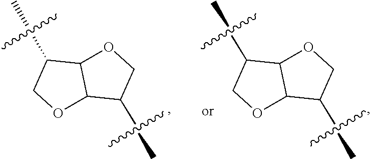
the at least one diacid is a compound of formula:
HO—(CO)—R₃—(CO)—OH, R₃ is C₂-C₁₂ alkylene,
the at least one amino acid is chosen from naturally occurring amino acids and non-naturally occurring amino acid.

2. The method as defined in claim 1, wherein the amino-acid based polymer is a poly (ester amide urea) comprising the following two blocks with random distribution thereof: 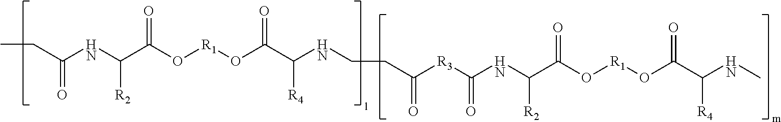
wherein
the ratio of l:m ranges from 0.05:0.95 to 0.95:0.05, l+m=1,
R₁ is chosen from C₂-C₁₂ alkylenes optionally interrupted by at least one oxygen, C₃-C₈ cycloalkylenes, C₃-C₁₀ cycloalkylalkylenes, 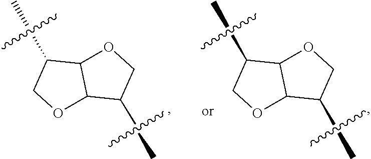
R₃ is C₂-C₁₂ alkylene,
R₂ and R₄ are independently chosen from the side chains of L- and D-amino acids so that the carbon to which R₂ or R₄ is attached has L or D chirality.

3. The method as defined in claim 1, wherein the polymer is in the form of polymer microcapsules.

4. The method as defined in claim 1, wherein the polymer is in the form of polymer nanocapsules.

5. The method as defined in claim 3, wherein the microcapsules are suspended in a liquid.

6. The method as defined in claim 1, wherein the composition is sprayed on the wound.

7. The method as defined in claim 1, wherein the biodegradable amino-acid based polymer is devoid of anti-bacterial agents.

8. The method as defined in claim 1, wherein the biodegradable amino-acid based polymer is devoid of bacteriophages.

9. The method as defined in claim 1, wherein the biodegradable amino-acid based polymer is essentially devoid of bacteriophages.

10. The method as defined in claim 1, wherein the polymer is in the form of a film.

11. The method as defined in claim 1, wherein the polymer is applied repeatedly on the wound.

12. The method as defined in claim 11, wherein the polymer is applied daily on the wound.

13. The method as defined in claim 12, wherein the polymer is applied until the wound reaches a predetermined healing status.

14. The method as defined in claim 1, wherein the wound is a chronic wound.

15. The method as defined in claim 14, wherein the chronic wound is selected from the group consisting of diabetic foot ulcers, pressure ulcers, venous stasis ulcers, and ischemic ulcers.

16. The method as defined in claim 1, wherein the composition further comprises at least one of an analgesic and an anti-inflamatory agent.

17. The method as defined in claim 1, wherein the composition further comprises at least one of ibuprofen, lidocaine, opioids, and cannabinoids.

18. The method as defined in claim 3, wherein the microcapsules consist essentially of the amino-acid based polymer.

19. The method as defined in claim 3, wherein the microcapsules consist of the amino-acid based polymer.

20. The method as defined in claim 4, wherein the nanocapsules consist essentially of the amino-acid based polymer.

21. The method as defined in claim 4, further comprising a permeation enhancer promoting permeation of the nanocapsules in the wound.

22. The method as defined in claim 1, wherein the composition further includes at least one additional component selected from the group consisting of: metal ions, zinc ions, silver ions, anti-microbial peptides (AMPs), Human Cathelicidin LL-37, Innate Defense Regulator 1018 peptide (IDR-1018), Human β-defensis (hBD-2 and hBD-3), Pexiganan, Tiger17 peptide, Growth factors (Gfs), granulocyte-macrophage colony-stimulating factor (GM-CSF), basic fibroblast growth factor (bFGF), platelet derived growth factor (PDGF), vascular endothelial growth factor (VEGF), stem cells, bone marrow-derived stem cells (BMSCs), adipose-derived stem cells (ADSCs), cellular adhesion molecules (CAMs), antibiotics or antimicrobials, analgesics, polyethylene glycol and anti-inflammatory agents.

23. The method as defined in claim 1, wherein the composition is part of a dressing selected from the group consisting of gauzes, transparent films, foams, hydrogels, hydrocolloids, and hydroconductive dressings.

24. The method as defined in claim 1, wherein the composition is dispersed in or covalently bonded to the remainder of the dressing.

25. The method as defined in claim 1, further comprising degrading the composition in tissues part of the wound or adjacent to the wound.

26. The method as defined in claim 1, wherein the composition is in the form of a coating layer coats an implantable device.

27. The method as defined in claim 26, wherein the coating layer is designed to completely biodegrade over of predetermined duration, the predetermined duration being one of at least one month and at least one year.

28. The method as defined in claim 1, wherein where R₁ is —(CH₂)₆—, R₃ is —(CH₂)₈—, and R₂ and R₄ are the side chain of L-leucine.
